(19) United States
(12) Patent Application Publication
(10) Pub. No.: US 2014/0016097 A1
(43) Pub. Date: Jan. 16, 2014

(54) Device and Method for Detecting Ophtalmic and/or Brain Diseases

(71) Applicants: Matteo Leonardi, Pully (CH); Jean-Marc Wismer, Lausanne (CH); Sacha Cerboni, Lausanne (CH); Mario Schlund, Ecublens (CH)

(72) Inventors: Matteo Leonardi, Pully (CH); Jean-Marc Wismer, Lausanne (CH); Sacha Cerboni, Lausanne (CH); Mario Schlund, Ecublens (CH)

(73) Assignee: SENSIMED SA, Lausanne (CH)

(21) Appl. No.: 14/003,124

(22) PCT Filed: Mar. 6, 2012
(86) PCT No.: PCT/EP2012/053825

§ 371 Date: Sep. 4, 2013

Related U.S. Application Data
(60) Provisional application No. 61/472,660, filed on Apr. 7, 2011.

(51) Int. Cl. A61B 3/10; A61B 5/00; A61B 3/113; A61B 3/16

(52) U.S. Cl. CPC A61B 3/10; A61B 3/16; A61B 5/6821; A61B 3/113
USPC 351/209; 600/398; 600/558; 351/205; 351/219; 351/246

(57) ABSTRACT

System for monitoring at least one biomechanical ophthalmic parameter, the system comprising a measuring device (1) adapted to be placed on or implanted into an eye of a patient for measuring said at least one biomechanical ophthalmic parameter of said eye, a recording device (6) for obtaining from said measuring device (1) measurement data representative of the instant value of said at least one biomechanical ophthalmic parameter of said eye, wherein said recording device is adapted to obtain said measurement data at a predetermined frequency equal to or greater than twice the variation frequency of said at least one biomechanical ophthalmic parameter. Method comprising monitoring at least one biomechanical ophthalmic parameter of an eye of a patient using a measuring device (1) adapted to be placed on or implanted into an eye of a patient for measuring the at least one biomechanical ophthalmic parameter of the eye, obtaining from the measuring device (1) a plurality of measurement data representative of the value the at least one biomechanical ophthalmic parameter of the eye measured at a predetermined frequency, wherein the predetermined frequency is equal or greater than twice the variation frequency of the at least one biomechanical ophthalmic parameter, at least partly automatically analyzing the plurality of measurement data in order to determine an ophthalmic condition of the eye.

### CROSS-REFERENCE TO RELATED APPLICATIONS

This is a 371 US national stage application of PCT/EP2012/053825, filed Mar. 6, 2013, and entitled “Device and Method for Detecting Ophtalmic and/or Brain Diseases” and benefit is claimed of U.S. Patent Application Ser. No. 61/472,660, filed Apr. 7, 2011, and entitled “Device and Methods for Detecting Opthalmic Diseases”.

### BACKGROUND OF THE INVENTION

The present invention relates to a device and methods for monitoring biomechanical ophthalmic parameters and/or for detecting and/or diagnosing ophthalmic diseases. Moreover, as the eyes are connected with the brain, the monitoring of these ophthalmic parameters, and in particular the monitoring of the intraocular pressure, can be used according to the invention for detecting and/or diagnosing brain diseases, like headache or intracranial hypertension for example. The present invention relates in particular to a system comprising a device that can be placed in the eye of a user in order to monitor a specific behavior, for example the eye blink pattern, the rapid eye motion pattern and/or the pulsation pattern, of one or more ophthalmic parameters including for example the intraocular pressure, over an extended period of time, for example during a specific activity, drug instillation, etc.

Devices for measuring the intraocular pressure (IOP) over a period of time are known in the art. These devices typically comprise a pressure sensor for continuously measuring the IOP, which is embedded for example into a contact lens that is placed in a non-invasive way on the patient's eye, or into a support that is implanted into the patient's eye. These devices further comprise a receiving unit and a telemetry system for acquiring IOP data from the sensor at given intervals over a period of time. The IOP values measured and recorded are for example averaged and/or filtered, if needed, and then interpreted by physicians in order to detect elevations of intraocular pressure that could lead to glaucoma, which conducts to a gradual loss of vision.

The systems described in the prior art are for example designed to measure a few IOP values per second during a few seconds and perform this measurement cycle every few minutes over a certain period of time, usually up to 24 h, in order to get the circadian or nycthemeral profiles of the IOP.

### SUMMARY OF THE INVENTION

It is an aim of the present invention to provide a system comprising a near real time measurement of biomechanical ophthalmic parameters, including for example, but not exclusively, the IOP profile, eye blink and/or rapid eye motion, with a high resolution.

It is another aim of the present invention to provide methods of computing and analyzing the recorded data in order to diagnose ophthalmic diseases such as for example, but not exclusively, glaucoma, posterior ischemic optic neuropathy, behavioral disorder, sleeping disorders, and/or in order to determine the suitable treatment and/or in order to follow up the patient and manage the disease with a tailored treatment.

These aims and other advantages are achieved with a system and methods according to the respective independent claims.

These aims are achieved in particular by a system for monitoring at least one biomechanical ophthalmic parameter, the system comprising a measuring device adapted to be placed on or implanted into an eye of a patient for measuring said at least one biomechanical ophthalmic parameter of said eye, a recording device for obtaining from said measuring device measurement data representative of the instant value of said at least one biomechanical ophthalmic parameter of said eye, wherein said recording device is adapted to obtain said measurement data at a predetermined frequency equal to or greater than twice the variation frequency of said at least one biomechanical ophthalmic parameter.

These aims are also achieved in particular by a method comprising monitoring at least one biomechanical ophthalmic parameter of an eye of a patient using a measuring device adapted to be placed on or implanted into an eye of a patient for measuring the at least one biomechanical ophthalmic parameter of the eye, obtaining from the measuring device a plurality of measurement data representative of the value of the at least one biomechanical ophthalmic parameter of the eye measured at a predetermined frequency, wherein the predetermined frequency is equal or greater than twice the variation frequency of the at least one biomechanical ophthalmic parameter, at least partly automatically analyzing the plurality of measurement data in order to determine an ophthalmic condition of the eye.

The system and the method of the invention thus allow continuously monitoring one or more ophthalmic parameters, for example the IOP, in various situations, for example during normal activities of a patient, before and after a particular event, etc., and with a high resolution, in order to allow a fine and reliable analysis of the condition of the eye.

In embodiments, the system of the invention comprises a very sensitive and accurate sensor, for example a pressure sensor, which allows achieving a precise and accurate measurement of the IOP. According to the invention, by maximizing the accuracy, the sensitivity and the frequency of IOP measurements, biomechanical parameters can be observed and measured, which could not be measured with prior art systems. The system and the methods of the present invention thus allow measuring, computing and analyzing biomechanical ophthalmic parameters like for example, but not exclusively, eye blink and/or pulsation patterns of intraocular pressure, eye movements during rapid eye motion phases, etc.

### BRIEF DESCRIPTION OF THE DRAWINGS

The invention will be better understood by reading the following description illustrated by the figures, where:

FIG. 1 shows a device for measuring biomechanical ophthalmic parameters over a period of time according to an embodiment of the invention.

FIG. 2 shows a system for monitoring biomechanical ophthalmic parameters and/or for detecting and/or diagnosing ophthalmic diseases according to an embodiment of the present invention, comprising the device of FIG. 1.

FIG. 3 shows an example of a biomechanical response of the eye to an eye blink stimulation.

FIG. 4 shows examples of responses of eyes in various conditions to an eye blink stimulation.

FIG. 5 shows an example of a pulsation pattern of the intraocular pressure.

FIG. 6 shows an example of a pattern of eye blinking during the awakening period.

FIG. 7 shows an example of a pattern of eye blinking over an extended period of time.

FIGS. 8a and 8b partially show a system for measuring rapid eye motion according to an embodiment of the invention.

FIG. 9 shows a typical pattern of rapid eye motion during a sleep period. FIG. 10A-D show example of variations of a monitored biomechanical ophthalmic parameter due to various events.

### DETAILED DESCRIPTION

In embodiments, the present invention relates to a device, a system and methods for measuring and/or monitoring one or more biomechanical ophthalmic parameter in order for example to determine the response of an eye of a patient to various events and/or situations including for example, but not exclusively, an eye blink stimulation, the pulsation of intraocular pressure, the rapid eye motion during a period of sleep, the use of drugs or medication, physical activity of the patient, etc., using a system capable of continuously and accurately measuring at least one biomechanical ophthalmic parameter, including for example, but not exclusively, intraocular pressure, corneal curvature and/or micro-displacement of the eye, with a frequency at least twice as high as the frequency of the changes of the at least one parameter to be measured, for example at least 10 Hz, over an extended period of time. In embodiments, the present invention further describes a system comprising a computer having preprogrammed algorithms, or a computer program thereon able to display, analyze and process the measured data and give for example essential information on the ophthalmic condition of the eye when the computer program is run on the computer.

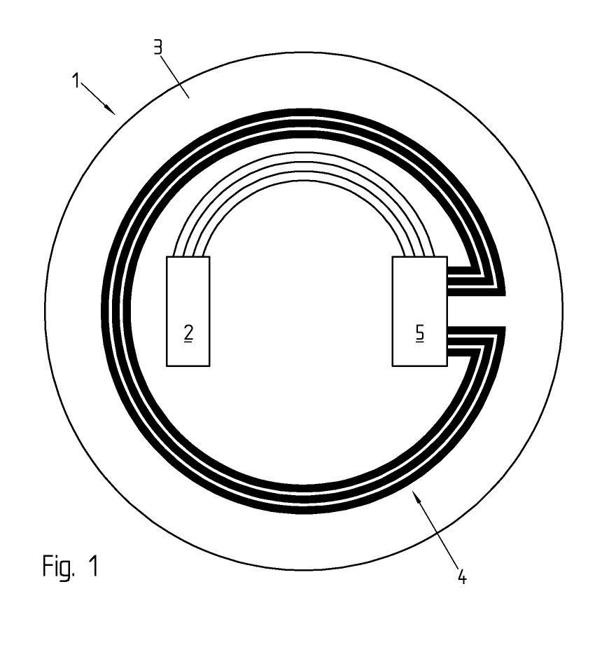

FIG. 1 schematically illustrates an example of a device 1 for measuring at least one biomechanical ophthalmic parameter over a period of time according to embodiments of the invention. The device 1 for example comprises at least one sensor 2, for example a pressure sensor, adapted for measuring a biomechanical ophthalmic parameter, for example the intraocular pressure (IOP). The sensor 2 is attached, preferably fixedly attached, to a support 3. The support 3 is adapted for placing the sensor 2 in direct or indirect contact with the eye of a patient in order to allow the sensor 2 measuring the corresponding parameter. In the illustrated embodiment, the support 3 is a contact lens, for example a soft contact lens, and the sensor 2 is for example embedded in the contact lens and positioned such that it is in direct or indirect contact with the surface of the eye when the device 1 is worn by a patient like a conventional contact lens.

In other embodiments, the device is an implantable device that can be implanted into the eye for measuring the at least one biomechanical ophthalmic parameter, the support being thus adapted for being implanted into the eye, using for example known surgical methods.

The sensor 2 is of any type adapted for measuring the at least one ophthalmic parameter. In the illustrated example, the sensor 2 is for example a pressure sensor in the form of a MEMS (Micro Electromechanical System), for example a piezoresistive or piezoelectric pressure sensor with a diaphragm and a pressure cavity that create a variable resistance for detecting strain due to pressure applied on the diaphragm. Other types of sensors, for example, but not exclusively, other types of pressure sensors, are however possible within the frame of the invention. In embodiments, the sensor is for example a pressure sensor using at least one active strain gage and at least one passive strain gage embedded into a support in the form of a contact lens, preferably a soft contact lens, which allows achieving a precise and accurate measurement of the IOP and/or of mechanical deformations of the eyeball.

In the illustrated embodiment, the device further comprises communication means 4, for example an antenna for allowing wireless communication from and/or to the device 1, and a microcontroller 5. The microcontroller 5 for example powers the sensor 2, reads measurement data from the sensor 2 that correspond to the value of the at least one measured parameter, optionally at least temporarily stores measurement data and/or sends measurement data over the communication means 4, for example wirelessly sends measurement data over the antenna, to an external device. In other embodiments, the communication means comprises wired communication means. The communication means 4 and the microcontroller 5 are preferably fixedly attached to the support 3, for example embedded in the support 3.

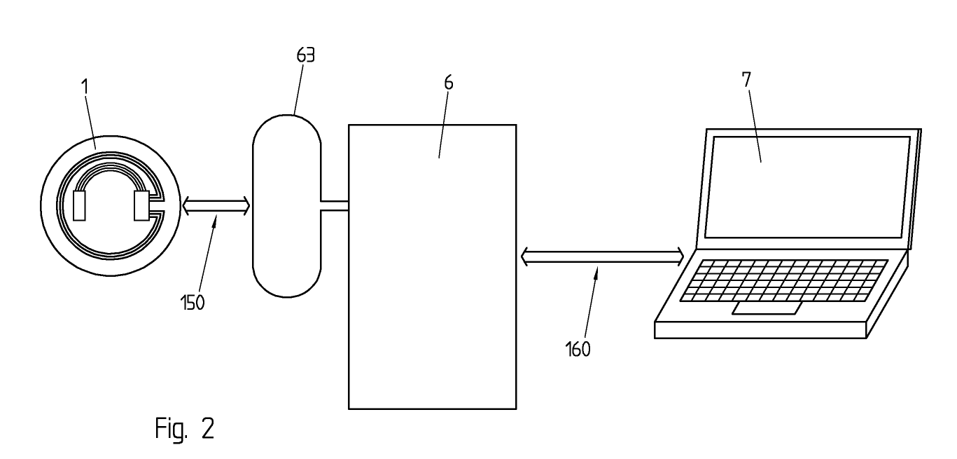

FIG. 2 schematically illustrates an example of a system for monitoring at least one biomechanical ophthalmic parameter and/or for detecting and/or diagnosing ophthalmic diseases, according to embodiments of the invention.

The system for example comprises a measuring device 1 as described above in relation with FIG. 1, for example in the form of a soft contact lens with a pressure sensor, a portable recording device 6 for communicating with the measuring device 1 and/or storing the collected information during the monitoring time periods, and a computing device 7, for example a computer, for storing, analyzing, computing and/or displaying the data collected and stored by the portable communication device 6.

The portable recording device 6 comprises a first communication interface for communicating with the pressure measuring device 1. The first communication interface is for example a wireless communication interface comprising an antenna 63 that is advantageously placed near the measuring device 1 when the measuring device 1 is worn by a user. The antenna 63 is for example integrated into eyeglasses, not represented on the figures, and/or into a for example disposable, flexible and hypoallergenic patch, also not represented on the figures, which are or is worn by the user during the monitoring time periods. Other means are however possible within the frame of the invention for placing the antenna 63 at a suitable distance from the measuring device 1 when the latter is worn by a user.

The portable recording device 6 further comprises a second communication interface for communicating with the computing device 7.

According to embodiments of the invention, when monitoring the at least one biomechanical ophthalmic parameter, a user wears the measuring device 1, for example by placing the support in the form of a contact lens on his or her eye just like any conventional contact lens or by having the device in an implantable form previously implanted in one of his or her eyes, and carries the portable recording device 6, for example in a pocket or by hanging it around his or her neck. The antenna 63 is placed as close as possible to the user's eye wearing the measuring device 1 in order to allow the establishment of a first communication channel 150, for example a wireless communication channel, between the measuring device 1 and the recording device 6. In case of wireless communication, the antenna 63 is preferably oriented in a plane as parallel as possible to the plane of the antenna of the measuring device 1 in order to allow for an efficient powering of the microcontroller and/or of the pressure sensor over the communication channel 150, which is for example a close distance inductive communication channel. The antenna 63 is for example integrated in eyeglasses and/or into a patch surrounding the eye, for example into a disposable, flexible and hypoallergenic patch, and/or in a cap, a hat or in another piece of clothing or accessory worn by the user. Preferably, the antenna 63 is centered with the antenna of the measuring device 1 when the measuring device 1 and the portable recording device 6 are both worn by the user. The diameter of the antenna 63 of the portable recording device 6 is preferably larger than the diameter of the measuring device 1. The shape of the antenna 63 of the portable recording device 6 is for example round, oval, rectangular, polygonal, or any other appropriate shape. The shape of the antenna 63 of the portable recording device 6 is preferably adapted to the shape of the device, for example the eyeglasses, the patch, the piece of garment, etc., to which it is attached.

According to embodiments, while monitoring the at least one biomechanical ophthalmic parameter, the portable recording device 6 powers the measuring device 1 through the first communication channel 150 at for example regularly spaced time intervals and collects data sent by the microcontroller for example through the antenna of the measuring device 1.

Collected data for example comprises electrical signals from the sensor and/or a value of the at least one monitored biomechanical ophthalmic parameter calculated by the microcontroller of the measuring device 1 on the basis for example of the sensor's electrical signals. In embodiments, the collected data is stored in internal memory of the portable recording device 6.

The at least one biomechanical ophthalmic parameter is for example measured at a predetermined frequency.

In embodiments, the predetermined measuring frequency is equal to or higher than twice the frequency of the variations of the at least one biomechanical ophthalmic parameter to be monitored. The predetermined frequency thus for example depends on the finality of the monitoring. The predetermined frequency for example depends on the known or supposed frequency of an event inducing a variation of the measured at least one biomechanical ophthalmic parameter.

In embodiments, the predetermined frequency is chosen to allow for a precise and detailed representation of the variations of the at least one biomedical ophthalmic parameter. The predetermined measuring frequency is thus for example in the range of 10 to 20 Hz in order to allow a precise representation of the variation of the at least one biomedical ophthalmic parameter in a short period of time, for example the variation of the parameter during a blink of the eye.

The at least one biomechanical ophthalmic parameter is for example measured at the predetermined frequency over an extended period of time, for example seconds, minutes or hours depending for example on the variations of the at least one parameter that need to be analyzed and/or on the diagnosis that needs to be made. In embodiments, the at least one biomechanical ophthalmic parameter is measured at the predetermined frequency for limited periods of time, for example some seconds or some minutes, wherein the limited measuring periods are repeated for example at regular intervals or upon triggering, for example upon occurrence of a particular event.

The method of the invention thus allows a precise monitoring of the variations of the at least one parameter over extended periods of time, including at night, while the user is asleep.

At some moments in time, for example once a day, once a week or once a month, the user and/or a practitioner connects the portable recording device 6 to a computing device 7, for example to a computer, over a second communication channel 160, for example a wireless communication channel, for example a Bluetooth, Wi-Fi or any other appropriate wireless communication channel. The second communication channel 160 can however also be any appropriate wired communication channel. Once the portable recording device 6 is connected to the computing device 7, the data collected and stored in the internal memory of the portable recording device 6 is transferred over the second communication channel 160 to the computing device 7 for further analysis, for example in order to detect and/or diagnose ophthalmic diseases, and/or in order to control the effects of a medical treatment followed during the monitoring period, determine its efficiency and/or possibly adapt it in case of need.

In embodiments, at least part of the data analysis and/or of the corresponding decisions are performed automatically with the help of one or more computer programs running on the computing device 7. The detection, diagnosis, control, determination and/or adaptation is performed in particular by at least partly automatically analyzing the variations of the at least one ophthalmic parameter measured during the monitoring period. In embodiments the measured variations over time are for example compared with typical variation schemes corresponding for example to that of a healthy or standard eye. Any significant difference between the measured scheme and the sample scheme is for example automatically detected and/or analyzed in order to possibly diagnose an ophthalmic disease. The measured values of the monitored at least one ophthalmic parameter and/or the typical values of said at least one ophthalmic parameter for a healthy or standard eye are for example displayed as one or more curves in a two-dimensional graph with the value of the at least one ophthalmic parameter being represented on the vertical axis and time on the horizontal axis.

The one skilled in the art will understand that, in the above and following examples, representing the value of a measured and/or monitored ophthalmic parameter, or depicting said value, on or by the axis of a graph can mean for example reporting on said axis a value of said ophthalmic parameter that was previously computed from at least one signal, for example an electrical signal, received from the corresponding sensor during the measuring and/or monitoring periods, or directly reporting on the axis the value of the electrical signal, which is representative of the value of the ophthalmic parameter to be monitored.

Similarly, analysing the measured values of the at least one ophthalmic parameter can mean analysing values of said parameter that were previously computed from the electrical signals received from the corresponding sensor, or analysing the values of the electrical signals received from the corresponding sensor.

In variant embodiments, the monitoring system comprises two measuring devices in order to allow simultaneously monitoring both eyes of a patient, for example over extended periods of time. Preferably, both measuring devices simultaneously and/or alternatively communicate with the same portable recording device 6 that for example is connected to and/or comprises two antennas. Accordingly, the portable recording device preferably stores or records data received from both intraocular measuring devices.

In an embodiment, the method of the invention for example allows measuring, displaying, analyzing and characterizing the response of an eye to an eye blink stimulation by monitoring the intraocular pressure (IOP) of the eye during at least one eye blink cycle and measuring, displaying, analyzing and/or characterizing the measured data. During a natural eye blink cycle, the eye lid is massaging the corneal surface of the eye and therefore causing biomechanical stimulations of the eye. These stimulations generate biomechanical responses inside the eye like modification of the corneal curvature and quick variation of the IOP. The responses are different depending on the ophthalmic conditions of the eye. According to the present invention, continuously and precisely measuring the response of the eye to an eye blink thus allows gathering useful indications on the ophthalmic condition of the eye and further helps diagnosing ophthalmic diseases.

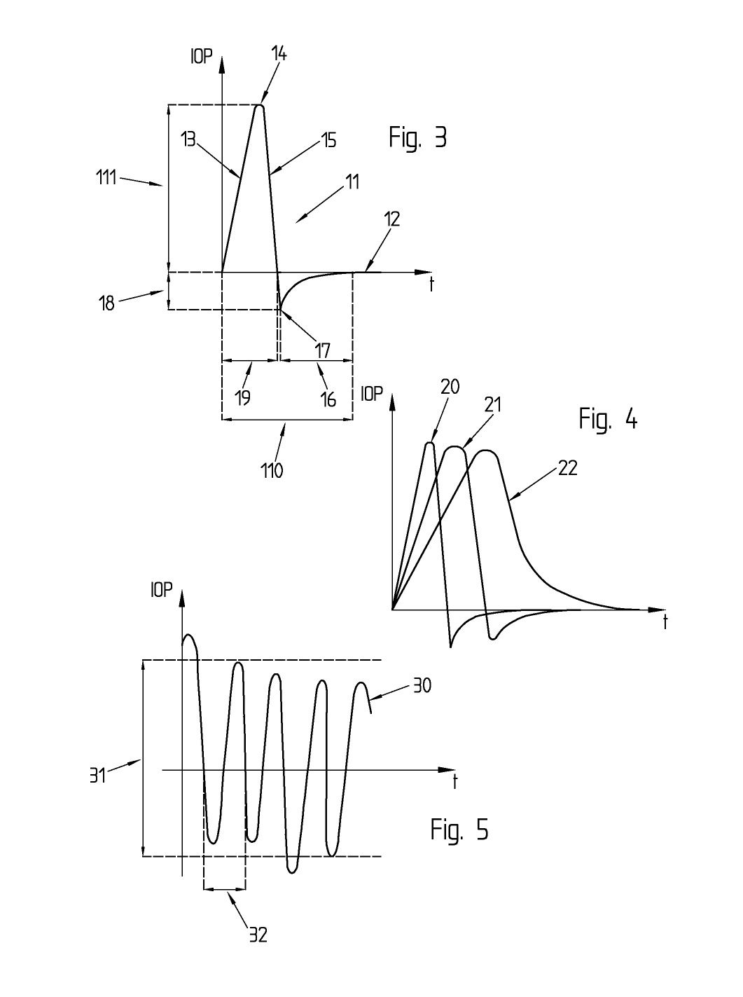

FIG. 3 shows a typical biomechanical response of an eye to an eye blink stimulation. This response is obtained by treating and displaying the values of intraocular pressure, measured by a measuring device according to the invention, in a two-dimensional graph where the horizontal axis depicts the time elapsed and where the vertical axis depicts the IOP value, for example directly or, as explained above, by reporting the value of an electrical signal, for example an electrical tension, obtained from a corresponding sensor and representative of the IOP.

Curve 11 shows a typical triphasic response of a healthy or standard eye. The resting pressure 12 is the intraocular pressure maintained in the eye as long as nothing perturbs it. The rising phase 13 is characterized by an abruptly shoot upward up to a peak value 14 which represents the maximal increase of intraocular pressure inside the eye caused by the eye lid pressure on the cornea during an eye blink stimulation. After reaching a peak value, relaxation of the intraocular tissues causes an abruptly decrease and stabilization of the intraocular pressure. This second phase is called the falling phase 15 where the intraocular pressure decreases to its initial value, often ending below its resting pressure 12 and remains for some period of time before gradually reaching back its resting pressure. This third and last phase is called the stabilization phase 16. The undershoot peak value 17 represents the minimal intraocular pressure inside the eye during an eye blink stimulation. The negative pressure interval 18 represents the total intraocular pressure drop, from the baseline resting pressure, occurring inside the eye during an eye blink stimulation. The positive pressure period 19 represents the period of time elapsed between the rising phase and the falling phase (limited to the time when it reaches its initial value). The positive pressure interval 111 represents the total intraocular pressure increase, from the baseline resting pressure 12, occurring inside the eye during an eye blink stimulation. The response period 110 represents the period of time between the rising phase 13 and the end of the stabilization phase 16.

It has been observed that the biomechanical response of the eye to an eye blink stimulation can differ under certain conditions. According to the present embodiment, the method of the invention is used for example to detect different pathologies of the eye by comparing the response to reference values. FIG. 4 shows three examples of eye responses that could be related to non-healthy eyes. Curve 20 shows a response where the negative pressure interval is big. Curve 21 shows a response where both positive pressure period and stabilization period are long. Curve 22 shows a response where the positive pressure period is very long and where there is no undershoot or negative pressure interval.

According to embodiments of the invention, the intraocular pressure (IOP) of the eye of a patient is thus for example monitored over a given period of time, for example during some seconds, some minutes or more, with a monitoring system as described above in relation with the example illustrated in FIG. 2, wherein the sensor of the measuring device 1 is a pressure sensor. The IOP values measured during a determined monitoring period are uploaded into the computing device 7 of the system and for example displayed as a curve representing the IOP variation over a time interval including at least one eye blink cycle. The variation of the measured IOP during the at least one eye blink cycle is analyzed, preferably automatically analyzed by said computing device 7 with the help of a corresponding computer program running on the computing device 7. The step of analyzing the measured data for example comprises automatically calculating and/or measuring the value of at least one indicator. According to the present embodiment, the at least one indicator is chosen from a group of indicators comprising for example the negative interval, the positive pressure period, the stabilization period and/or the negative pressure interval of the IOP variation during an eye blink cycle. The calculated and/or measured indicator values are then compared, for example automatically compared by the computing system 7, for example with typical values of corresponding indicators for a healthy eye. In case a significant difference is detected, for example automatically calculated by the computing system 7, between the value of at least one indicator of the monitored eye and a target value, or target value range, of the corresponding indicator, a message indicative of a possible non-healthy condition of the monitored eye is generated and possibly displayed by the computing device 7. In embodiments, the computing system 7 further automatically analyzes the noted difference or differences and automatically determines the condition of the monitored eye possibly responsible for such differences, for example a high intraocular pressure condition.

In a variant embodiment, the computing device 7 performs the above calculations and analysis over several eye blink cycles in order to confirm or invalidate the analysis and/or diagnosis performed on the basis of the IOP variations during a first eye blink cycle.

In an another embodiment, the method of the invention for example allows measuring, displaying, analyzing and characterizing the variation pattern of the intraocular pressure during the sleep and/or between eye blinks during the day. Accordingly, the intraocular pressure of an eye of a patient is monitored during several hours during the day and/or the night. In addition to the intraocular pressure pulsations due to the heart beat, which happens at the same frequency as the heartbeat, the monitoring system and method of the invention allow measuring that other pulsations of the intraocular pressure occur during some periods of sleep and/or during the day. These other intraocular pressure pulsations have higher amplitude and thus a lower frequency than the pulsations due to the heartbeat. They are generated by the supply of blood to the optical nerve. Continuously and precisely measuring the pattern of these other pulsations during the sleep of a patient, and/or during the day between the eye blink cycles, thus provides useful indications on the ophthalmic condition of the eye and further helps diagnosing ophthalmic diseases like for example the posterior ischemic optic neuropathy.

FIG. 5 shows a typical pulsation pattern of the intraocular pressure over several heartbeats and between eye blink cycles, i.e. either during the night while the patient is asleep, or during the day between eye blink cycles. This pattern is obtained by displaying the values of intraocular pressure measured by the sensor embedded in the measuring device as a two-dimensional graph where the horizontal axis depicts the time elapsed and where the vertical axis depicts the IOP values. The scatter graph 30 shows a pulsation pattern having average pulsation amplitudes 31 and a pulsation frequency given by the time interval between two pulses 32. By comparing the pulsation amplitude and the pulsation frequency with baseline values, a neuropathic optic ischemia can be detected and treated in advance.

According to the present invention, the IOP values measured during an extended period of time, for example during the night while the patient is asleep, are uploaded into the computing device 7 and at least partly automatically analyzed and compared for example with typical values of a healthy eye for automatic detection of significant differences, automatic determination of the condition responsible for the detected differences, and/or automatic diagnosis of a pathology related to the determined condition.

In still another embodiment, the method of the invention for example allows measuring, analyzing and characterizing the patterns, and more precisely the frequency and the amplitude distribution over the monitoring time, of eye blink cycles during wake time. During an eye blink cycle, the eye lid is massaging the corneal surface of the eye and therefore causing biomechanical stimulations of the eye. These stimulations affect the corneal curvature and the intraocular pressure inside the eye which can be continuously measured and analyzed by the system of the invention. According to the invention, the computing device for example has preprogrammed algorithms, computer programs, for analyzing and/or displaying the measured data and/or for helping diagnosing ophthalmic and/or brain diseases.

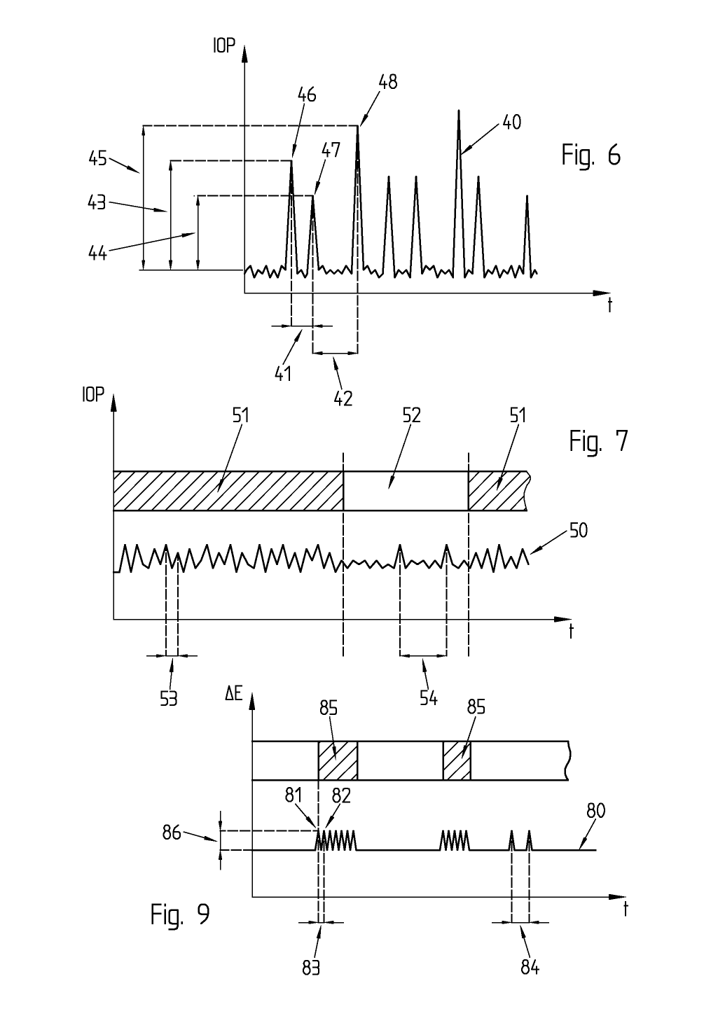

FIG. 6 shows a typical pattern of eye blink cycles of a patient during wake time, measured for example during the day. This pattern is obtained by displaying the values of intraocular pressure measured by a pressure sensor embedded in the measuring device of the system of the invention in the form of a two-dimensional graph where the horizontal axis depicts the time elapsed and where the vertical axis depicts the IOP profile. The scatter graph 40 shows a pattern having different peaks, each one representing a blink of the eye. A first eye blink characterized by a first peak 46 has a first blink strength represented by its amplitude 43. A second eye blink characterized by a second peak 47 has a second blink strength represented by its amplitude 44. The elapsed time 41 between the first peak 46 and the second peak 47 characterizes the frequency of the eye blinking. According to embodiments of the invention, the blink amplitudes and frequencies are for example determined automatically by the computing device of the system of the invention, averaged and compared with baseline values and/or they are used to trigger any abnormal activities of the eye. The graphical representation of the eye blink pattern shows a qualitative and quantitative distribution of the eye blink of a patient. It can be monitored during different periods of the day, when the patient is performing different activities under different conditions. The collected IOP data will then be at least partly automatically processed by the processing device of the system and for example displayed in order to provide valuable information for example to a physician and help in diagnosing ophthalmic and/or cerebral diseases. The graphical representation of the eye blink pattern is for example also used to analyze behavioral disorder of a patient during certain activities.

In further embodiments, the method of the invention allows measuring, analyzing and characterizing the awakening and sleeping period patterns using the eye blinking measurements.

FIG. 7 shows a typical pattern of eye blinking cycles over an extended period of time. This pattern is obtained by displaying with the computing device of the monitoring system of the invention the values of intraocular pressure measured by the sensor embedded in the measuring device in a two-dimensional graph where the horizontal axis depicts the time elapsed and where the vertical axis depicts the IOP profile. The scatter graph 50 shows a pattern having different peaks, each peak representing a blink of the eye. The computing device having preprogrammed algorithms, computer programs, analyzes and/or displays the measured data. Accordingly, the computing device is for example programmed to automatically determine that when the average elapsed time 53 between two successive eye blinks is short or under a threshold value, the subject is awake, and that when the average elapsed time 54 between two successive eye blinks raises above a threshold value, the subject is asleep. In a further step, the lengths of awakening periods 51 and sleeping periods 52 are measured in order for example to automatically diagnose sleeping disorders, for example in case of very short sleeping periods or other predetermined symptoms.

In other embodiments, the system and method of the invention for example allow measuring, analyzing and characterizing the rapid eye motion patterns of a subject. During sleep, rapid eye motion (REM) is a normal stage of sleep characterized by rapid movements of the eyes. REM sleep in adult humans typically occupies 20-25% of total sleep, about 90 to 120 minutes of a night of sleep. During a normal night of sleep, human beings usually experience about four or five periods of REM sleep which are relatively short at the beginning of the night and longer towards the end. During REM, the activity of the brain's neurons is quite similar to that during waking hours; for this reason, the REM sleep stage may be called paradoxical sleep. Being able to analyze and characterize the REM may be very beneficial for patients with suspected sleeping disorders.

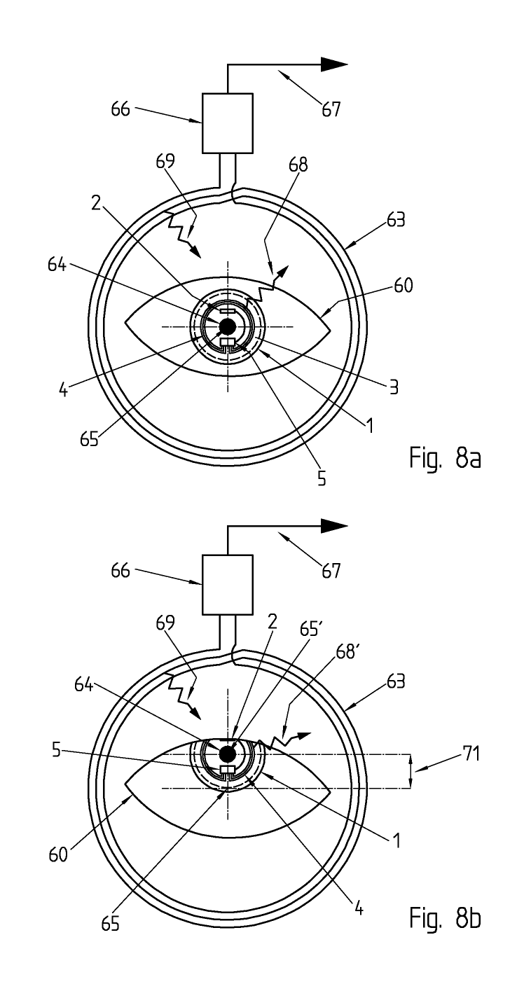

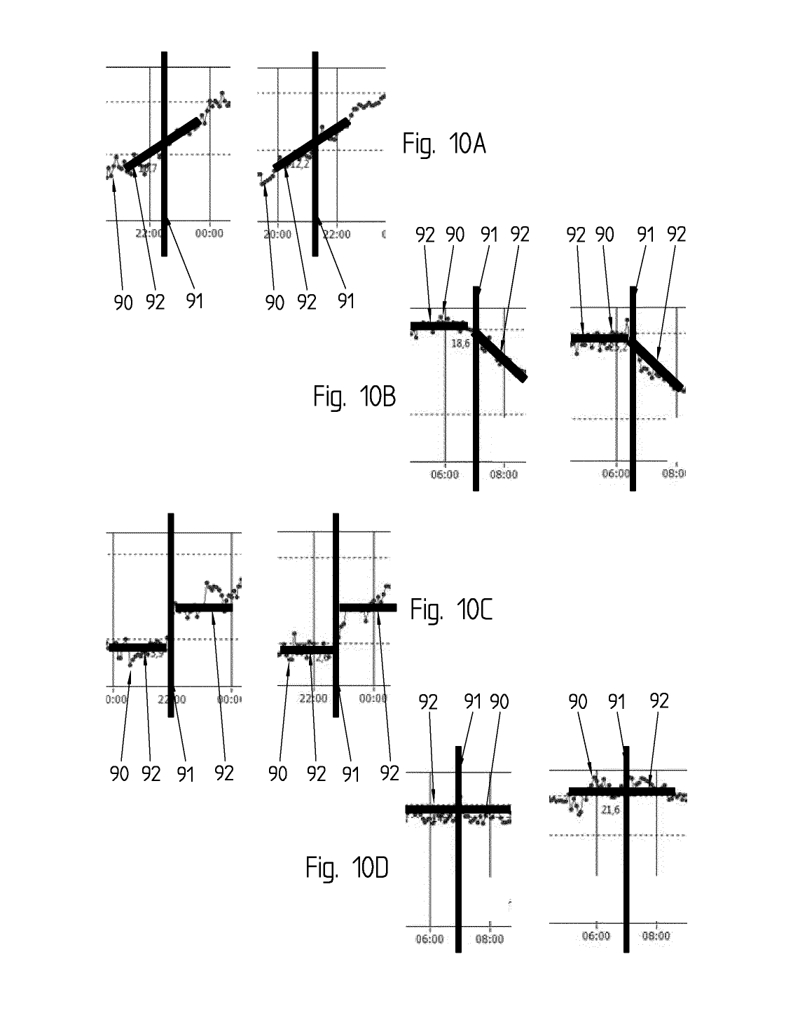

FIG. 8a schematically illustrates an eye 60 wearing a measuring device 1 of the system of the invention in the form of a contact lens, which is centered on the pupil 64 of the eye 60. The measuring device 1 comprises at least one sensor 2, for example a pressure sensor, adapted for measuring a biomechanical ophthalmic parameter, for example the intraocular pressure (IOP), a microcontroller 5 and a secondary coil antenna 4 embedded in the support 3, for example a soft contact lens. The microcontroller 5 for example converts the analogical measurements of sensor to digital data and for example wirelessly transmits them over the antenna 4 to a recording device following an appropriate communication protocol.

The recording device is preferably located at a short distance of the eye, for example in the form of an external eye patch placed around the eye of the subject, and/or at least partly integrated into or attached to eyeglasses. The recording device further comprises an antenna 63, for example a coil antenna, a microcontroller 66 and a second communication interface 67 for communicating for example to a computing device. Transmission of commands 69 are sent from the antenna 63 of the recording device and received by the antenna 4 of the measuring device 1. Electrical energy is transmitted from the recording device to the measuring device 1 through electro-magnetic induction. An electric current flowing through the primary coil antenna 63 creates a magnetic field that acts on the secondary coil antenna 4 of the measuring device 1, therefore producing a current within it. As the distance from the primary coil antenna 63 increases, and/or the relative alignment between the primary coil antenna 63 and the secondary coil antenna 4 decreases, more and more of the magnetic field misses the secondary coil antenna 4 and therefore reduces the amount of inductive energy. The amount of inductive energy received by the secondary coil antenna 4 of the measuring device 1 is measured by the microcontroller 5, converted to digital data 68 and transmitted back to the recording device along with the data generated by the pressure sensor embedded in the measuring device 1. As shown in FIG. 8b, any movement of the eye produces a displacement 71 of the measuring device 1. This displacement 71 induces a displacement and/or a misalignment of the secondary coil antenna 4 relative to the primary coil antenna 63 and therefore reduces the amount of energy induced in the measuring device 1. This new amount of energy is measured by the microcontroller 5, converted to digital data 68 and transmitted back to the recording device. The variation of energy induced in the measuring device 1 is directly proportional to the amplitude of the movement of the eye and can be further communicated by the wireless recording device to the computing device and used to at least partly automatically analyze and quantify the rapid eye motion and/or the amplitude of the eye movement with respect to its centered position.

FIG. 9 shows a typical pattern of rapid eye motion during the sleep period. This pattern is obtained, for example automatically generated by the computing device of the monitoring system of the invention, by displaying the values representing the variation of energy induced in the measuring device in a two-dimensional graph where the horizontal axis depicts the time elapsed and where the vertical axis depicts the variation of energy induced in the measuring device. The curve 80 shows a pattern having different peaks, each one representing a movement of the eye. A first eye motion characterized by a first peak 81 has a relative displacement represented by the amplitude of the variation of energy 86 induced in the measuring device 1. A second eye motion characterized by a second peak 82 has a relative displacement represented by the amplitude of the variation of energy induced in the measuring device 1. The elapsed time 83 between the first peak 81 and the successive second peak 82 characterizes the frequency of the rapid eye motion. According to the invention, the computing device having preprogrammed algorithms analyzes and displays the measured data, and determines that a REM period 85 starts when the average elapsed time 83 between two successive movements of the eye is short or under a threshold value, and that the REM period 85 ends when the average elapsed time 84 between two successive movements of the eye raise above a threshold value. The computing device preferably graphically displays the REM in order to show a qualitative and quantitative distribution of the REM of a patient, thus providing valuable information for example to a physician and help in diagnosing ophthalmic and/or brain diseases and/or sleep disorders.

In embodiments, and with reference to FIG. 10A to 10D, the method and the system of the invention are used for measuring the changes induced in at least one biomechanical ophthalmic parameter by a particular event, such as for example, but not exclusively, the patient waking up, falling asleep, changing position, taking in a particular substance or undergoing a change in its blood pressure. Accordingly, the at least one biomechanical ophthalmic parameter is measured with the measuring device of the invention at a predetermined frequency over a period going from a certain time before the event to another time after the event. The measured values are then treated and for example displayed as a two-dimensional graph where the horizontal axis depicts the time elapsed and where the vertical axis depicts the measured biomechanical ophthalmic parameter or a value representative of it.

FIGS. 10A to 10D illustrate various examples of possible changes of a biomechanical ophthalmic parameter monitored according to the invention, due to the occurrence of a particular event. In the figures, the curve 90 represents the actually measured values representative of the monitored parameter and the thick vertical line 91 illustrates the occurrence of a particular event at a particular time. The thick horizontal and/or oblique line that at least partly overlaps the curve 90 represents the slope 92 of the variation of the value of the monitored parameter, i.e. the variation of an averaged value of the monitored parameter, before and/or after the event.

FIGS. 10A-D show examples of possible schemes for the variation of the monitored parameter due to a particular event, i.e. possible slopes 92. The variation is for example a continuous and regular change of the parameter value before and after the particular. The slope 92 is thus continuous before and after the event and can be either an ascending (FIG. 10A) or a descending slope. According to another scheme, the variation is a stepped change of the monitored parameter value (FIG. 10C), whereas the slope 92 is a stepped curve that has a constant value before the event, and another constant value, either higher or lower, after the event. In still another scheme, the variation is a continuous change of the monitored parameter value after the particular event only. The slope 92 is thus horizontal before the event and either ascending or descending (FIG. 10B) after the event. In still another scheme, there is no significant variation of the parameter before or after the event, such that the slope 92 remains constant before and after the event (FIG. 10D).

Accordingly, by analyzing, for example at least partly automatically, a measured variation scheme, in particular a computed slope 92, it is possible to determine and possibly quantify the effect of a particular event on a monitored parameter value. For example, in case of a delay between the occurrence of the event and an expected variation of the slope, or in case of an unexpected variation of the slope, a condition of the eye on which the parameter was measured can be determined, thus allowing for example diagnosing a particular condition of the eye, measuring the evolution of a treatment, etc. For example, determining and analyzing the slope of a particular parameter around the event of going to sleep/waking up, changing body position, etc., is of great interest in order to assess the physiology of the eye and its capability to adapt to a changing condition. Absent or reduced adaptation capability can be for example an indication of a potential pathological behavior.

In variant embodiments, the method and the system of the invention are used for monitoring the long term evolution of at least one biomedical ophthalmic parameter, for example in order to evaluate the effectiveness of a medical treatment and/or in order to evaluate the mid- to long-term effects of a drug on the at least one biomedical ophthalmic parameter. Accordingly, the at least one biomedical ophthalmic parameter is measured continuously or at intervals during and/or after the medical treatment and/or drug application period. The values of the at least one biomedical ophthalmic parameter measured during the latest measuring period are compared, for example at least partly automatically compared, with previously measured values of the parameter, thereby allowing determining, for example at least partly automatically determining, a positive, negative or neutral evolution of the measured parameter over time, for example over days, weeks, months or years.

In applications of the present invention for the diagnosis and/or treatment of a patient having an ophthalmic and/or a brain disease, for example, and/or in applications for the measurement of the effects of a substance and/or of an event on a measured ophthalmic parameter, several of the above described methods can be combined in order to obtain for example, but not exclusively, a more reliable diagnosis, a better follow up of a medical treatment and/or a more accurate knowledge of the effects of external elements on at least one biomechanical ophthalmic parameter.

The above embodiments of the system and methods of the invention are illustrative and in no way limiting examples of the present invention. In particular, the invention is contemplated to encompass all variations of constructions, wherein a measuring device, a monitoring system and methods of measurements are used to measure the response of the eye to an eye blink stimulation, the pulsation of intraocular pressure and the rapid eye motion, etc. In embodiments, the system of the invention is configured for continuously and accurately monitoring one or more biomechanical ophthalmic parameters, for example intraocular pressure, corneal curvature and/or micro-displacement of the eye, at a frequency of at least 10 Hz during an extended period of time, for example several hours. According to the invention, the monitoring system comprises computing means, for example a computer, having preprogrammed algorithms able to display, analyze and process the data measured during the monitoring periods and provide essential information on the ophthalmic condition of the eye and/or diagnose ophthalmic and/or brain diseases.

Therefore, the principles and features of the present invention may be employed in various and numerous embodiments without departing from the scope of the invention. In particular, any combination of the above-described embodiments of the method is possible within the frame of the invention.

1. System for monitoring at least one biomechanical ophthalmic parameter, said system comprising:
a measuring device (1) adapted to be placed on or implanted into an eye of a patient for measuring said at least one biomechanical ophthalmic parameter of said eye;
a recording device (6) for obtaining from said measuring device (1) measurement data representative of the instant value of said at least one biomechanical ophthalmic parameter of said eye;
wherein said recording device is adapted to obtain said measurement data at a predetermined frequency equal to or greater than twice the variation frequency of said at least one biomechanical ophthalmic parameter.

2. System according to claim 1, wherein said predetermined frequency is equal or greater than 10 Hz.

3. Method comprising:
monitoring at least one biomechanical ophthalmic parameter of an eye of a patient using a measuring device (1) adapted to be placed on or implanted into an eye of a patient for measuring said at least one biomechanical ophthalmic parameter of said eye;
obtaining from said measuring device (1) a plurality of measurement data representative of the value said at least one biomechanical ophthalmic parameter of said eye measured at a predetermined frequency, wherein said predetermined frequency is equal or greater than twice the variation frequency of said at least one biomechanical ophthalmic parameter;
at least partly automatically analyzing said plurality of measurement data in order to determine an ophthalmic condition of said eye.

4. Method according to claim 3, further comprising at least partly automatically ascertaining a correspondence of said ophthalmic condition to an identification of an ophthalmic disease.

5. Method according to claim 3, wherein said monitoring is performed during at least 24 hours in order to obtain at least one circadian or nycthemeral profile of said at least one biomechanical ophthalmic parameter.

6. Method according to claim 3, wherein said determination of an ophthalmic condition of said eye comprises diagnosing an ophthalmic pathology.

7. Method according to claim 3, further comprising comparing said ophthalmic condition of said eye with a previously determined ophthalmic condition of said eye in order to determine an evolution of said ophthalmic condition.

8. Method according to claim 7, further comprising adjusting a medical treatment on the basis of said evolution.

9. Method according to claim 3, wherein the plurality of measured data are displayed as a two-dimensional shape that graphically depicts the variations of said at least one biomechanical ophthalmic parameter over time.

10. Method according to claim 9, wherein the two-dimensional shape comprises:
a first portion representing a peak and a vertical variation of the at least one biomechanical ophthalmic parameter; and
a second portion representing a settling time of the at least one biomechanical ophthalmic parameter.

11. Method according to claim 10, wherein a magnitude of the vertical variation of the biomechanical ophthalmic parameter comprises a percentage of the peak.

12. Method according claim 10, wherein a magnitude of the vertical variation of the biomechanical ophthalmic parameter comprises an absolute value.

13. Method according to claim 3, wherein variations of the at least one biomechanical ophthalmic parameter are caused by the natural eyeblink of the eye.

14. Method according to claim 3, wherein variations of the at least one biomechanical ophthalmic parameter are caused by a specific activity of said patient.

15. Method according to claim 3, wherein variations of the at least one biomechanical ophthalmic parameter are caused by a substance externally or internally applied to said patient.

16. Method according to claim 3, wherein the at least one biomechanical ophthalmic parameter comprises deformation of the eye.

17. Method according to claim 3, wherein the at least one biomechanical ophthalmic parameter comprises the intraocular pressure.

18. Method according to claim 3, wherein the at least one biomechanical ophthalmic parameter comprises the position of the eye.

19. Method according to claim 3, wherein the measuring device (1) comprises a sensor (2) embedded in a support (3) in the form of a contact lens.

20. Method according to claim 3, wherein the measuring device (1) comprises a sensor (2) embedded in a support (3) adapted to be implanted into said eye.

21. Method according to claim 3, comprising:
continuously monitoring said at least one biomechanical ophthalmic parameter during a sleep period;
outputting a plurality of measured data from said measuring device (1) determined from a pulsation pattern of intraocular pressure; and
determining a correspondence of an amplitude of the pulsation, a frequency of the pulsation and a duration of the pulsation to an identification of an ophthalmic and/or brain disease.

22. Method according to claim 3, wherein said predetermined frequency is equal or greater than 10 Hz.

23. Method according to claim 3, wherein continuously monitoring comprises measuring said at least one biomechanical ophthalmic parameter at said predetermined frequency during limited measuring periods that are repeated during a time cycle.

24. Method according to claim 23, wherein said limited measuring periods last thirty seconds and are repeated every 5 minutes within a time cycle.

25. Method according to claim 23, wherein said limited measuring periods are repeated upon triggering.

26. Method according to claim 3, wherein analyzing said plurality of measured data comprises measuring a strength value of the eyeblinks and a frequency of the eyeblinks over predetermined periods of time.

27. Method according to claim 3, wherein continuously monitoring comprises continuously monitoring the activity of eyeblinks of the patient over a 24-hour monitoring period of time including wake periods and sleep periods.

28. Method according to claim 27, further comprising determining a duration of the sleep periods and wake periods, and determining a number of eyeblinks that occur during the sleep periods.

29. Method according to claim 28, further comprising determining a duration of the sleep periods and wake periods, and determining the duration of rapid eye movement (REM) that occurs during the sleep periods and the wake periods.

30. Method according to claim 29, further comprising determining a number of REM episodes that occur during the sleeping period.

31. Method according to claim 3, wherein said step of continuously monitoring extends from a first instant before a particular event to a second instant after said particular event, and wherein said step of analysing comprises determining a scheme of a variation of said at least one parameter due to said particular event.

32. Method according to claim 3, further comprising the step of detecting and/or diagnosing brain diseases on the basis of said determined ophthalmic condition of said eye.

33. Method according to claim 32, wherein said brain diseases comprise headaches or intracranial hypertension.
